(19) United States
(12) Patent Application Publication
(10) Pub. No.: US 2005/0031655 A1
(43) Pub. Date: Feb. 10, 2005

(54) Emulsion composition

(75) Inventor: Inna Karpov, Germantown, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Plough Healthcare Products, Inc.

(21) Appl. No.: 10/633,985

(22) Filed: Aug. 4, 2003

(51) Int. Cl. A61K 7/42

(52) U.S. Cl. 424/401; 424/59

(57) ABSTRACT

An emulsion composition for skin application comprises an aqueous component, one or more water-insoluble organic components totaling at least about 15 percent by weight of the composition, and about 2 to about 10 percent by weight of a solid component consisting of porous silica microspheres having an average particle size between about 5 μm and about 20 μm.

### INTRODUCTION TO THE INVENTION

The present invention relates to an emulsion composition for skin application, and more particularly to an emulsion composition that contains porous silica microspheres.

Sensory characteristics of emulsion compositions that contain high amounts of water-insoluble organic ingredients tend to discourage product use, as consumers prefer to apply products that seem to be more aqueous in nature. However, highly occlusive skin care products that alleviate skin dryness and help to heal conditions such as skin cracking generally are required to contain large concentrations of very oily substances, such as mineral oil, lanolin, petrolatum, and others. Also, the more effective sunscreen products frequently use as their active ingredients high concentrations of organic ultraviolet-absorbing compounds that have an oily character. Such skin care and sunscreen products feel greasy, sticky, or oily when spread on the skin and this sensation cannot readily be reduced by even a prolonged rubbing of the product into the skin.

It is desired to obtain emulsion products that contain high concentrations of functional water-insoluble organic ingredients, and also provide the desirable non-oily skin sensations that are obtained with products containing low concentrations of the functional ingredients.

### SUMMARY OF THE INVENTION

In one aspect, the invention includes an emulsion composition for skin application comprising an aqueous component, one or more water-insoluble organic components totaling at least about 15 percent by weight of the composition, and about 2 to about 10 percent by weight of a solid component consisting of porous silica microspheres having an average particle size between about 5 μm and about 20 μm.

The invention also encompasses sunscreen compositions containing at least about 12 percent by weight of one or more water-insoluble organic ultraviolet-absorbing active ingredients, and about 2 to about 10 percent by weight of a solid component consisting of porous silica microspheres having an average particle size between about 5 μm and about 20 μm.

Particularly when the total amount of water-insoluble organic components in an emulsion composition exceeds about 20 or about 25 weight percent, the skin feel improvement benefits of the invention will be quite dramatic. Thus, the emulsion composition will preferably contain at least about 20, and more preferably about 25, weight percent of water-insoluble organic components.

### DETAILED DESCRIPTION

In the following description and the claims, it is intended that a reference to a percentage means percent by weight, unless the context clearly indicates otherwise. Since the chemical names for certain composition ingredients are quite cumbersome, some ingredients are identified herein by their adopted names as given in standard reference works, including J. A. Wenninger et al., Eds., International Cosmetic Ingredient Dictionary and Handbook, 8^{th} Ed., The Cosmetic, Toiletry, and Fragrance Association, Washington, D.C., 1999.

Emulsions are generally considered to be dispersions of micro droplets of nonaqueous fluid substances in a bulk aqueous phase (“oil-in-water” emulsions) or dispersions of micro droplets of an aqueous phase in a bulk nonaqueous fluid phase (“water-in-oil” emulsions). Lotions have a liquid character, while creams are similar emulsions that are more semi-solid. Either type of emulsion can also contain dispersed particulate substances. More complex emulsion compositions are also known, in which an emulsion is itself dispersed in an aqueous or nonaqueous fluid phase; these less frequently encountered compositions are called oil-in-water-in-oil, or water-in-oil-in-water, emulsions. Compositions having an aqueous bulk (or “external”) phase tend to give more pleasing skin sensations, when they are applied, and the following discussion will focus on oil-in-water emulsions even though the invention is also useful for producing the other types.

After emulsions are applied to the skin, the water evaporates, leaving behind the contained water-soluble, insoluble solid, and nonvolatile organic “oil” components. Rubbing the emulsion during its application tends to hasten water evaporation and causes a fraction of the residual components to more readily enter pores and other discontinuities in the skin surface. However, if the emulsion contains high concentrations of oily or greasy ingredients, or is applied in a large amount, the sensations during rubbing will not be pleasing to the user and an oily, greasy, or sticky residue may remain on the skin surface.

By use of the present invention a greatly reduced sensation of oiliness, greasiness, and stickiness can be obtained, even when the emulsion contains a large proportion of “oil” ingredients constituting the nonaqueous phase. This beneficial property results from inclusion in the emulsion of about 1 to about 10 percent by weight of porous silica microspheres having an average particle size between about 5 μm and about 20 μm. Typically, the silica microspheres will be present in amounts about 2 to about 8 percent by weight, or in amounts about 3 to about 6 weight percent.

The silica microspheres that are useful in this invention are commercially available from multiple sources, and include those sold by Kobo Products, Inc. of South Plainfield, N.J. U.S.A. and identified as MSS-500W. That product is described in the manufacturer's literature as a white spherical particle powder of at least 98 percent purity, having a particle size range of 2 μm to 20 μm as measured by a light scattering particle sizer, an average particle size between 10 μm and 14 μm as measured with a Coulter Counter Model TA-II, and a specific surface area between 700 m²/g and 900 m²/g as determined by single point BET technique. Sunjin Chemical Co. Ltd. of Ansan City, KyungKido, Korea sells spherical porous silica beads identified as SUNSIL™ 130 and having an average particle size of 6 μm to 9 μm. Porous spherical silica beads having a surface area of 400 m²/g to 600 m²/g are sold by U.S. Cosmetics Corporation of Dayville, Conn. U.S.A. with the designation SB-700. A number of additional products not mentioned here, but having a similar composition and properties, are also useful in the invention.

All of the porous silica microspheres have the capability of absorbing large amounts of oils, which property could adversely affect their usefulness when the emulsion product contains organic ultraviolet-absorbing ingredients, since an unpredictable release from the microspheres of active ingredients after skin application of the product can prevent prolonged maintenance of sunscreening activity, particularly when the skin is in contact with water. Thus, it is preferable to either pre-treat the silica microspheres with water or an aqueous solution of product ingredients before the microspheres are incorporated into the emulsion product, or to include the silica microspheres in the aqueous component mixture during the formulation procedure. This will tend to fill the pores with aqueous material and prevent significant subsequent oil absorption.

The emulsion of the present invention typically contains one or more emulsifiers and water; and may optionally contain one or more ingredients that are emollients, humectants, dry-feel agents, preservatives, antioxidants, chelating agents, and fragrances, as well as sunscreen active agents, waterproofing agents, dyes, and any other class of materials whose presence may be cosmetically, efficaciously, or otherwise desirable.

Emulsifiers

Typical suitable emulsifiers suitable for forming oil-in-water emulsions, and therefore having HLB values between about 1 and about 7, include sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-distearate, polyacrylamide (and) C₁₃-C₁₄ isoparaffin (and) laureth-7, polyglycerol esters of oleic/isostearic acid, polyglyceryl-6 hexaricinolate, polyglyceryl-4 oleate, polyglyceryl-4 oleate/PEG-8 propylene glycol cocoate, arachidyl alcohol (and) benehyl alcohol (and) arachidyl glucoside, oleamide DEA, sodium glyceryl oleate phosphate and hydrogenated vegetable glycerides phosphate.

Other emulsifiers useful in the present invention may be non-ionic, liquid or solid at room temperature and preferably compatible, i.e., soluble and stable, with emollients. Preferred emulsifiers for oil-in-water emulsions usually have a HLB value of less than about 5, e.g., sorbitan sesquioleate (HLB value 3.7), sorbitan monooleate (HLB value 4.3) and sorbitan trioleate (HLB value 1.8). Other preferred emulsifiers include polymeric emulsifiers such as copolymers of C₁₀-C₃₀ alkyl acrylates and one or more monomers of acrylic acid or methacrylic acid, also known as Pemulen® TR1 and TR2, trademark of B. F. Goodrich Inc., Cincinnati, Ohio. Other emulsifiers include sorbitan esters such as sorbitan isostearate available as Crill 6, tradename of Croda Inc. of New York, N.Y.; polyglyceryl-3 distearate available as Cremophor, tradename of tradename of BASF, Parsippany N.J.; and carbomer, which is a homopolymer of acrylic acid crosslinked with an allyl ether of sucrose, available as Carbopol 941, tradename of B.F. Goodrich, Cleveland, Ohio; and surfactants such as such as DEA-cetyl phosphate, also known as Amphisol®, trademark of Bernel Chemical Co., Englewood, N.J.

During preparation of the emulsion, an acid or a base may be added to adjust the pH of one or more ingredients, e.g. to adjust the viscosity of a polymeric thickener, prior to its inclusion in the emulsion composition. For example, triethanolamine, a base, can be used to increase the pH of the water phase and consequently, modify the desired viscosity of the emulsion. An emulsifier can also be formed by an acid-base or other reaction, such as results from including both triethanolamine and stearic acid as ingredients in the composition.

Conveniently, one or more emulsifiers can be used in the compositions of the present invention in amounts ranging from about 0.05 to about 20 weight percent of emulsion, preferably from about 0.1 to about 15 percent, more preferably from about 5 to about 10 percent.

Water

Water is employed in amounts effective to form the emulsion. For hydrophilic or water-loving ingredients, e.g., emulsifiers, emollients, etc., the amount of water should be sufficient to at least solubilize these ingredients. For hydrophobic or water-repelling ingredients, the water should be employed in amounts to serve as the continuous phase of the emulsion, for oil-in water emulsions. Thus, amount of water in the emulsion or composition can range from about 2 to 95 weight percent, preferably from 50 to 85 weight percent. Purified water is preferred, to obtain more predictable product properties.

Emollients

An emollient is an oleaginous or oily substance which helps to smooth and soften the skin, and may also reduce its roughness, cracking, or irritation. Typical suitable emollients include mineral oil, for example a mineral oil having a viscosity in the range of 50 to 500 centipoise (0.05 to 0.5 Pa·s), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil, and peanut oil.

Other suitable emollients include squalane, castor oil, polybutene, odorless mineral spirits, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, isopropyl laurate, octyl palmitate which is commercially available as Lexol EHP, tradename of Inolex Co. of Philadelphia, Pa., isopropyl myristate, hexadecyl stearate, butyl stearate, decyl oleate, acetyl glycerides, the octanoates and benzoates of C₁₂-C₁₅ alcohols, the octanoates and decanoates of alcohols and polyalcohols such as those of glycol and glycerol, ricinoleates of alcohols and poly alcohols such as those of isopropyl adipate, hexyl laurate, and octyl dodecanoate.

Other suitable emollients which are solids or semi-solids at ambient temperatures may be used in amounts sufficient to provide liquid topical compositions. Such solid or semi-solid cosmetic emollients include hydrogenated lanolin, hydroxylated lanolin, acetylated lanolin, petrolatum, isohexadecane, isopropyl lanolate, butyl myristate, cetyl myristate, myristyl myristate, myristyl lactate, cetyl alcohol, isostearyl alcohol, and isocetyl lanolate. One or more emollients can optionally be included in the emulsion in an amount up to about 50 weight percent, preferably about 5 to about 40 weight percent.

Humectants

A humectant is a moistening agent that promotes retention of water due to its hygroscopic properties. Suitable humectants include glycerin, polymeric glycols such as poyethylene glycol, and polypropylene glycol, and sorbitols such as sorbitol solution. One or more humectants can optionally be included in the in the emulsion in amounts up to 10 weight percent, preferably about 1 to about 10 weight percent.

Dry-feel Modifier

A dry-feel modifier is an agent which when added to a emulsion, imparts a “dry feel” to the skin when the emulsion dries. Dry-feel modifiers may also reduce sunscreen migration on the skin. Dry feel modifiers can include, without limitation: starches; talc; kaolin; chalk; zinc oxide; silicone fluids; inorganic salts such as barium sulfate and sodium chloride; C₆ to C₁₂ alcohols such as octanol; sulfonated oils; surface treated silica, precipitated silica, fumed silica such as Aerosil® available from the Degussa Inc. of New York, N.Y., or mixtures thereof; or dimethicone, a mixture of methylated linear siloxane polymers, available as DC200 fluid, tradename of Dow Corning, Midland, Mich. One or more dry-feel modifiers can optionally be included in the emulsion in amounts up to about 20 weight percent, preferably from about 0.5 to about 6 weight percent.

Antimicrobial Preservatives

An antimicrobial preservative is a substance or preparation which destroys, prevents or inhibits the multiplication/growth of microorganisms in the emulsion composition and may offer protection from oxidation. Preservatives are used to make self-sterilizing, aqueous based products such as emulsions. This is done to prevent the development of microorganisms that may be in the product from growing during manufacturing and distribution of the product and during use by consumers who may inadvertently contaminate the products during normal use. Typical preservatives include: the lower alkyl esters of p-hydroxybenzoates (parabens), especially methylparaben, propylparaben, isobutylparaben, and mixtures thereof; diazolidinyl urea; benzyl alcohol; and benzoic acid. One or more antimicrobial preservatives can optionally be included in the emulsion composition in an amount up to about 10 weight percent, preferably about 0.05 to about 2 percent.

Antioxidants

An antioxidant is a natural or synthetic substance added to the emulsion to protect from or delay its deterioration due to the action of oxygen in the air (oxidation). Anti-oxidants prevent oxidative deterioration which may lead to the generation of rancidity and nonenyzymatic browning reaction products, and may act to protect the skin against free radical damage. Typical suitable antioxidants include propyl, octyl and dodecyl esters of gallic acid, butylated hydroxyanisole (BHA, usually as a mixture of ortho and meta isomers), butylated hydroxytoluene (BHT), various botanical extracts such as from green tea, white tea, grape seed, phyllanthus emblica, and the like, nordihydroguaiaretic acid, vitamin E, vitamin E acetate, vitamin C, and alkylated parabens such as methylparaben and propylparaben. One or more antioxidants can optionally be included in the emulsion composition in amounts up to about 5 weight percent, preferably about 0.05 to about 2 percent.

Chelating Agents

Chelating agents are substances used to chelate or bind metallic ions with a certain heterocylic ring structure so that the ion is held by chemical bonds from each of the participating ring. Suitable chelating agents include ethylene diaminetetraacetic acid (EDTA), EDTA disodium, calcium disodium edetate, EDTA trisodium, EDTA tetrasodium, and EDTA dipotassium. One or more chelating agents can optionally be included in the emulsion in amounts up to about 0.1 weight percent.

Fragrances

Fragrances are aromatic compounds that can impart an esthetically pleasing aroma to the emulsion composition. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the emulsion composition in amounts up to about 10 weight percent, preferably about 0.05 to about 5 percent.

Sunscreen Actives

The compositions of the present invention can contain a sunscreening effective amount of one or more oil-soluble or water-soluble sunscreening UV-B actives and optionally one or more UV-A actives. UV-A type sunscreening actives absorb long wavelength actinic radiation of the sun in the 320 to 400 nm range and UV-B type sunscreening actives absorb shorter wavelength actinic radiation in the 290-320 nm range. As is well known, particulate inorganic sunscreening actives can reflect or disperse a wide range of wavelengths, generally including both of the UV-A and UV-B ranges.

Typical sunscreen actives that are currently approved for use in the United States include: aminobenzoic acid up to 15 weight percent; avobenzone up to 3 weight percent; cinoxate up to 3 weight percent; dioxybenzone up to 3 weight percent; ensulizole up to 4 weight percent; homosalate up to 15 weight percent; meradimate up to 5 weight percent; octinoxate up to 7.5 weight percent; octisalate up to 5 weight percent; octocrylene up to 10 weight percent; oxybenzone up to 6 weight percent; padimate O up to 8 weight percent; sulisobenzone up to 10 weight percent; titanium dioxide up to 25 weight percent; trolamine salicylate up to 12 weight percent; and zinc oxide up to 25 weight percent. Other active ingredients and amounts are permitted in other countries, and their use is also contemplated for the present invention.

One or more sunscreen actives can be employed in the emulsion composition, usually in amounts totaling at least about 10 weight percent and generally totaling up to about 50 weight percent, typically about 10 to about 35 weight percent of the emulsion composition. A combination of two or more sunscreen active ingredients frequently will be present if ultraviolet absorption is desired for the composition.

Waterproofing Agents

A waterproofing agent is a hydrophobic material that imparts film forming and waterproofing characteristics to an emulsion. Typical suitable waterproofing agents include copolymers from polymerization of octadecene-1 and maleic anhydride in accordance with the published procedures such as those in U.S. Pat. No. 3,860,700 and U.S. Reissue Pat. No. 28,475. A preferred waterproofing agent is a polyanhydride resin, also known as PA-18, trade name of the Chevron Chemicals Co., San Francisco, Calif. Another preferred waterproofing agent is a copolymer of vinyl pyrollidone and eicosene monomers such as Ganex Polymer, trade name of ISP Inc. of Wayne, N.J.

Sunscreening products that contain waterproofing agents are described as being “water resistant” or “very water resistant” depending on whether the applied product remains effective after 40 or 80 minutes of water immersion, according to tests published by the United States Food and Drug Administration as Title 21, Code of Federal Regulations, Section 352.76.

One or more waterproofing agents can optionally be included in the composition in an amount ranging up to about 10 weight percent, preferably about 1 to about 10 weight percent.

Other Ingredients

As is known in the art, the foregoing categories of ingredients are not the only substances that can be advantageously incorporated into a product. For purposes of product esthetics or for therapeutic reasons, additional ingredients that are vitamins, provitamins, skin protectants, collagen, elastin, and others will frequently be included. Components that have other cosmetic purposes, including sunless tanning agents such as dihydroxyacetone, can also be incorporated into the compositions of the invention; one very useful application of the invention is to form esthetically pleasing products containing both a sunless tanning agent and a high-SPF producing amount of organic sunscreen ingredients.

In addition to these cosmetic ingredients, the emulsions of the invention can be prepared to contain pharmaceutical therapeutic ingredients such as, without limitation thereto, corticosteroids, antihistamines, topical anesthetics, antibacterials, antivirals, antimycotics, analgesics, antineoplastics, keratolytics, and others.

Containers

The emulsions of the present invention can be stored or dispensed in any container suitable for convenient delivery, i.e. pouring or spraying. Such containers can include but are not limited to jars, tubes, bottles with or without lotion pumps, pump spray bottles, and pressurized aerosol canisters. For convenience and economy, the container will frequently be formed from a thermoplastic substance, by molding.

### EXAMPLE 1

The effect of silica microspheres on the sensory characteristics of an occlusive skin treatment are evaluated. Mixtures of petrolatum and silica microspheres (as the commercial product MSS-500W from Kobo Products, Inc., South Plainfield, N.J. U.S.A.) are prepared by mixing the components to produce silica concentrations of 5 wt. percent and 10 wt. percent. Quantities amounting to 2 mg of these mixtures, and of petrolatum that contains no silica, are each spread onto 1 cm² of a subject's forearm skin and rubbed into the skin. The subject then evaluates the treated skin areas for the presence of stickiness and greasiness, and reports a noticeable decline in both of those sensations for the silica-containing compositions, the sensations being less objectionable as the silica concentration increased.

While this mixture is not an emulsion, it is considered useful as a demonstration of the beneficial effect of the microspheres in an extremely greasy and sticky matrix.

### EXAMPLE 2

A sunscreen lotion having an SPF value of 45 and a pleasant, non-greasy skin feel is prepared using the following ingredients:

| Ingredient | Grams |
|---|---|
| Part A | |
| Water | 49.85 |
| Acrylates/C10-30 alkylacrylate crosspolymer# | 0.3 |
| Part B | |
| Propylene glycol | 5 |
| Soluble collagen | 0.01 |
| Hydrolyzed elastin | 0.01 |
| DL-Panthenol | 0.01 |
| Disodium EDTA | 0.01 |
| Phyllanthus emblica | 0.1 |
| Silica microspheres* | 5 |
| Triethanolamine | 0.35 |
| Sodium ascorbyl phosphate | 0.01 |
| Part C | |
| PVP/eicosene copolymer | 2.5 |
| Stearic acid | 0.5 |
| Polyglyceryl-3 distearate | 0.29 |
| Methylparaben | 0.3 |
| Sorbitan isostearate | 0.71 |
| Homosalate | 15 |
| Oxybenzone | 6 |
| Octisalate | 5 |
| Propylparaben | 0.1 |
| Dimethicone | 0.4 |
| Avobenzone | 2 |
| Octocrylene | 5 |
| Part D | |
| Vitamin A palmitate | 0.01 |
| DL-α-Tocopherol | 0.5 |
| Part E | |
| Benzyl alcohol | 1 |
| Fragrance | 0.04 |

#PEMULEN ™ TR-2 from B. F. Goodrich Performance Materials, Cleveland, Ohio U.S.A.
*MSS-500W from Kobo Products, Inc., South Plainfield, New Jersey U.S.A.

An aqueous mixture is prepared by adding the PEMULEN TR-2 to the water and mixing to obtain a clear solution, then adding the Part B ingredients, in the order given, with continuous mixing.

A nonaqueous mixture is prepared by combining Part C ingredients, with continuous mixing, then commencing heating with continued mixing to obtain a temperature between about 60° C. and about 63° C., then cooling to about 49° C. Add the Part D ingredients.

An emulsion is formed by adding the heated nonaqueous mixture to the aqueous mixture, with vigorous mixing. Then the ingredients of Part E are combined and added to the emulsion and the emulsion is allowed to cool, with continuous mixing. When the emulsion reaches ambient temperature, water is added as required to make a total composition weight of 100 grams.

This product, when applied to the skin, does not impart a greasy or oily feeling, even though its nonaqueous content is high.

### EXAMPLE 3

A sunscreen lotion having an SPF value of 30 and a pleasant, non-greasy skin feel is prepared using the following ingredients:

| Ingredient | Grams |
|---|---|
| Part A | |
| Water | 54.39 |
| Acrylates/C10-30 alkylacrylate crosspolymer# | 0.3 |
| Part B | |
| Propylene glycol | 5 |
| Soluble collagen | 0.01 |
| Hydrolyzed elastin | 0.01 |
| DL-Panthenol | 0.01 |
| Disodium EDTA | 0.01 |
| Phyllanthus emblica | 0.1 |
| Silica microspheres* | 5 |
| Triethanolamine | 0.35 |
| Sodium ascorbyl phosphate | 0.01 |
| Part C | |
| PVP/eicosene copolymer | 2 |
| Stearic acid | 0.5 |
| Polyglyceryl-3 distearate | 0.29 |
| Methylparaben | 0.3 |
| Sorbitan isostearate | 0.71 |
| Homosalate | 15 |
| Oxybenzone | 5 |
| Octisalate | 5 |
| Propylparaben | 0.1 |
| Dimethicone | 0.4 |
| Avobenzone | 2 |
| Octocrylene | 2 |
| Part D | |
| Benzyl alcohol | 1 |
| Vitamin A palmitate | 0.01 |
| DL-α-Tocopherol | 0.5 |

#PEMULEN ™ TR-2 from B. F. Goodrich Performance Materials, Cleveland, Ohio U.S.A.
*MSS-500W from Kobo Products, Inc., South Plainfield, New Jersey U.S.A.

An aqueous mixture is prepared by adding the PEMULEN TR-2 to the water and mixing to obtain a clear solution, then adding the Part B ingredients, in the order given, with continuous mixing.

A nonaqueous mixture is prepared by combining Part C ingredients, with continuous mixing, then commencing heating with continued mixing to obtain a temperature between about 60° C. and about 63° C.

An emulsion is formed by adding the heated nonaqueous mixture to the aqueous mixture, with vigorous mixing. Then the ingredients of Part D are combined and added to the emulsion and the emulsion is allowed to cool, with continuous mixing. When the emulsion reaches ambient temperature, water is added as required to make a total composition weight of 100 grams.

This product, when applied to the skin, does not impart a greasy or oily feeling, even though its nonaqueous content is high.

### EXAMPLE 4

A sunscreen lotion having an SPF value of 30 and a pleasant, non-greasy skin feel is prepared using the following ingredients:

| Ingredient | Grams |
|---|---|
| Part A | |
| Water | 54.35 |
| Acrylates/C10-30 alkylacrylate crosspolymer# | 0.3 |
| Part B | |
| Propylene glycol | 5 |
| Soluble collagen | 0.01 |
| Hydrolyzed elastin | 0.01 |
| DL-Panthenol | 0.01 |
| Disodium EDTA | 0.01 |
| Phyllanthus emblica | 0.1 |
| Silica microspheres* | 5 |
| Triethanolamine | 0.35 |
| Sodium ascorbyl phosphate | 0.01 |
| Part C | |
| PVP/eicosene copolymer | 2 |
| Stearic acid | 0.5 |
| Polyglyceryl-3 distearate | 0.29 |
| Methylparaben | 0.3 |
| Sorbitan isostearate | 0.71 |
| Homosalate | 15 |
| Oxybenzone | 5 |
| Octisalate | 5 |
| Propylparaben | 0.1 |
| Dimethicone | 0.4 |
| Avobenzone | 2 |
| Octocrylene | 2 |
| Part D | |
| Vitamin A palmitate | 0.01 |
| DL-α-Tocopherol | 0.5 |
| Part E | |
| Benzyl alcohol | 1 |
| Fragrance | 0.04 |

#PEMULEN ™ TR-2 from B. F. Goodrich Performance Materials, Cleveland, Ohio U.S.A.
*MSS-500W from Kobo Products, Inc., South Plainfield, New Jersey U.S.A.

An aqueous mixture is prepared by adding the PEMULEN TR-2 to the water and mixing to obtain a clear solution, then adding the Part B ingredients, in the order given, with continuous mixing.

A nonaqueous mixture is prepared by combining Part C ingredients, with continuous mixing, then commencing heating with continued mixing to obtain a temperature between about 60° C. and about 63° C.

An emulsion is formed by adding the heated nonaqueous mixture to the aqueous mixture, with vigorous mixing. Then the ingredients of Part D are combined and added to the emulsion and the emulsion is allowed to cool, with continuous mixing. When the emulsion reaches ambient temperature, water is added as required to make a total composition weight of 100 grams.

This product, when applied to the skin, does not impart a greasy or oily feeling, even though its nonaqueous content is high.

### EXAMPLE 5

A sunscreen lotion having an SPF value of 15 and a pleasant, non-greasy skin feel is prepared using the following ingredients:

| Ingredient | Grams |
|---|---|
| Part A | |
| Water | 57.85 |
| Acrylates/C10-30 alkylacrylate crosspolymer# | 0.3 |
| Part B | |
| Propylene glycol | 5 |
| Soluble collagen | 0.01 |
| Hydrolyzed elastin | 0.01 |
| DL-Panthenol | 0.01 |
| Disodium EDTA | 0.01 |
| Phyllanthus emblica | 0.1 |
| Silica microspheres* | 5 |
| Part C | |
| PVP/eicosene copolymer | 2 |
| Stearic acid | 0.5 |
| Polyglyceryl-3 distearate | 0.29 |
| Methylparaben | 0.3 |
| Sorbitan isostearate | 0.71 |
| Homosalate | 12 |
| Sodium ascorbyl phosphate | 0.01 |
| Oxybenzone | 3.5 |
| Octisalate | 5 |
| Propylparaben | 0.1 |
| Dimethicone | 0.4 |
| Avobenzone | 2 |
| Octocrylene | 2 |
| Part D | |
| Vitamin A palmitate | 0.01 |
| DL-α-Tocopherol | 0.5 |
| Part E | |
| Water | 1 |
| Triethanolamine | 0.35 |
| Part F | |
| Benzyl alcohol | 1 |
| Fragrance | 0.04 |

#PEMULENTM ™ TR-2 from B. F. Goodrich Performance Materials, Cleveland, Ohio U.S.A.
*MSS-500W from Kobo Products, Inc., South Plainfield, New Jersey U.S.A.

An aqueous mixture is prepared by adding the PEMULEN TR-2 to the water and mixing to obtain a clear solution, then adding the Part B ingredients, in the order given, with continuous mixing.

A nonaqueous mixture is prepared by combining Part C ingredients, with continuous mixing, then commencing heating with continued mixing to obtain a temperature between about 60° C. and about 63° C., then cooling to about 43° C. and adding the Part D ingredients.

An emulsion is formed by adding the heated nonaqueous mixture to the aqueous mixture, with vigorous mixing. Then the ingredients of Parts E and F are added to the emulsion and the emulsion is allowed to cool, with continuous mixing. When the emulsion reaches ambient temperature, water is added as required to make a total composition weight of 100 grams.

This product, when applied to the skin, does not impart a greasy or oily feeling, even though its nonaqueous content is high.

### EXAMPLE 6

An occlusive skin care lotion is prepared from the following ingredients:

| Ingredient | Grams |
|---|---|
| Part A | |
| Water | 50.04 |
| Acrylates/C10-30 alkylacrylate crosspolymer# | 0.3 |
| Part B | |
| Propylene glycol | 5 |
| Disodium EDTA | 0.01 |
| Silica microspheres* | 5 |
| Triethanolamine | 0.35 |
| Part C | |
| PVP/Eicosene copolymer | 2.5 |
| Stearic acid | 0.5 |
| Polyglyceryl-3 distearate | 0.29 |
| Methylparaben | 0.3 |
| Sorbitan isostearate | 0.71 |
| Propylparaben | 0.1 |
| Dimethicone | 0.4 |
| Light mineral oil | 33 |
| DL-α-Tocopherol | 0.5 |
| Part D | |
| Benzyl alcohol | 1 |

#PEMULEN ™ TR-2 from B. F. Goodrich Performance Materials, Cleveland, Ohio U.S.A.
*MSS-500W from Kobo Products, Inc., South Plainfield, New Jersey U.S.A.

An aqueous mixture is prepared by adding the PEMULEN TR-2 to the water and mixing to obtain a clear solution, then adding the Part B ingredients, in the order given, with continuous mixing.

A nonaqueous mixture is prepared by combining Part C ingredients, with continuous mixing, then commencing heating with continued mixing to obtain a temperature between about 60° C. and about 63° C., then cooling to about 43° C.

An emulsion is formed by adding the heated nonaqueous mixture to the aqueous mixture, with vigorous mixing. Then the ingredient of Part D is added to the emulsion and the emulsion is allowed to cool, with continuous mixing. When the emulsion reaches ambient temperature, water is added as required to make a total composition weight of 100 grams.

This product, when applied to the skin, does not impart a greasy, sticky, or oily feeling, even though its nonaqueous content is high.

### EXAMPLE 7

An occlusive skin care lotion is prepared from the following ingredients:

| Ingredient | Grams |
|---|---|
| Part A | |
| Water | 49.94 |
| Acrylates/C10-30 alkylacrylate crosspolymer# | 0.3 |
| Part B | |
| Propylene glycol | 5 |
| Disodium EDTA | 0.01 |
| Silica microspheres* | 5 |
| Triethanolamine | 0.35 |
| Part C | |
| PVP/Eicosene copolymer | 2.5 |
| Stearic acid | 0.5 |
| Polyglyceryl-3 distearate | 0.29 |
| Sorbitan isostearate | 0.71 |
| Petrolatum | 8.5 |
| Dimethicone | 0.4 |
| Light mineral oil | 25 |
| DL-α-Tocopherol | 0.5 |
| Part D | |
| Benzyl alcohol | 1 |

#PEMULEN ™ TR-2 from B. F. Goodrich Performance Materials, Cleveland, Ohio U.S.A.
*MSS-500W from Kobo Products, Inc., South Plainfield, New Jersey U.S.A.

A lotion is formed, using the same procedure as in the immediately preceding example. This product, when applied to the skin, does not impart a greasy, sticky, or oily feeling, even though its nonaqueous content is high.

### EXAMPLE 8

A sunless tanning lotion having a non-oily feel is prepared using the following ingredients:

| Ingredient | Grams |
|---|---|
| Part A | |
| Water | 58.7 |
| Propylene glycol | 5 |
| Sodium chloride | 0.5 |
| Diazolidinyl urea | 0.1 |
| Dihydroxyacetone | 5 |
| Part B | |
| Caramel color | 1.8 |
| Silica microspheres* | 3 |
| Part C | |
| Cyclopenatsiloxane (and) dimethicone copolyol | 6 |
| Polyglyceryl-4 isostearate (and) cetyl | 1 |
| dimethicone copolyol (and) hexyl laurate | |
| Isohexadecane | 10.5 |
| Cyclomethicone | 7.8 |
| DL-α-Tocopherol | 0.05 |
| Fragrance | 0.15 |
| Mica (and) titanium dioxide | 0.4 |

*MSS-500W from Kobo Products, Inc., South Plainfield, New Jersey U.S.A.

The water of Part A is added to a mixing vessel and the other Part A components are added, in the listed order, with continuous mixing. Then the Part B components are added and mixed to obtain a uniform dispersion.

In a separate container, the Part C components are combined and mixed. With continuous slow stirring, the aqueous mixture is added at a rate sufficiently slow to permit emulsion formation during the addition. After all of the aqueous material has been added, the stirring rate is rapidly increased to a high level, to form a stable emulsion.

### EXAMPLE 9

The sensory characteristics of sunscreening compositions according to preceding examples of the invention are evaluated, using the sensory descriptive analysis method described in Morten Meilgaard et al., Sensory Evaluation Techniques, CRC Press, Inc., Boca Raton, Fla. U.S.A., 1987. The panel consists of nine members who have been trained to conduct the analysis method, and each analysis is conducted twice for each sample, in two separate sessions. Testing involves dispensing 0.5 mL portions of a sample onto the center of a 5 cm diameter circle marked on an evaluator's forearm, then having the evaluator spread the sample within the circle with the index or middle fingers using rotational movements, at a rate of two strokes per second. Wetness and spreadability are evaluated after three rubs, thickness after twelve rubs, and oiliness, waxiness, and greasiness evaluated after fifteen to twenty rubs. Absorbency is evaluated by counting the number of rubs required for loss of a wet feeling on the skin surface, up to a maximum of 120 rubs.

For each measured attribute, evaluators use a 0-100 point scale, with a rating of 0 indicating an absence of the property and a rating of 100 indicating a very strong presence of the property. These grading scales are “anchored” using samples of commercially available products: a commercial baby oil is considered to have a rating of 35 for wetness, while petrolatum rates 22, talc rates 0 and water rates 100; for the amount of product residue on the skin surface immediately after applying, bare skin is rated 0 and petrolatum is rated 85. The baby oil is used as the anchor for slow absorption, as it requires more than 120 rubs to completely soak into the skin. A commercially available moisturizing skin lotion having no sunscreen component is evaluated in certain tests together with the inventive compositions, for benchmarking purposes.

Results are obtained, as follows:

I. Immediately After Product Application

| Attribute | Example 3 | Example 5 | Example 2 |
|---|---|---|---|
| Residue Thickness | 10.9 | 8.3 | 6.3 |
| Residue Amount | 12.5 | 9.9 | 7.8 |
| Percent Oily | 18.5 | 10.8 | 9.1 |
| Percent Waxy | 52.3 | 65.3 | 51.3 |
| Percent Greasy | 20.8 | 17.3 | 21.3 |
| Percent Silicone | 7.8 | 4.8 | 12.2 |

II. At Product Rub-Out

| | | | | Commercial |
|---|---|---|---|---|
| Attribute | Example 3 | Example 5 | Example 2 | Skin Lotion |
| Wetness | 63.4 | 59.1 | 56.2 | 69.0 |
| Spreadability | 69.0 | 65.1 | 58.8 | 69.0 |
| Product Thickness | 28.1 | 30.1 | 30.7 | 26.0 |
| Absorbency | 55.9 | 36.0 | 58.2 | 64.0 |

III. Twenty Minutes After Product Application

| | | | | Commercial |
|---|---|---|---|---|
| Attribute | Example 3 | Example 5 | Example 2 | Skin Lotion |
| Residue Thickness | 5.5 | 3.9 | 2.5 | 17 |
| Residue Amount | 6.4 | 5.4 | 3.6 | 13 |
| Percent Oily | 2.5 | 1.3 | 2.5 | N.D. |
| Percent Waxy | 57.0 | 57.3 | 38.8 | N.D. |
| Percent Greasy | 6.8 | 3.8 | 5.0 | N.D. |
| Percent Silicone | 7.5 | 5.0 | 3.1 | N.D. |

N.D. = Not determined

The inventive compositions are determined to have a wet feel at rub out, with high spreadability and low film thickness. All of the inventive compositions absorb into the skin more rapidly than the commercial skin lotion. Immediately after application, the inventive products give a very low perception of residue, indicating that the compositions provide a feel approximating that of untreated skin, the feel of the small residue of composition being described as predominately waxy, similar to hardened candle wax. Twenty minutes after application, residue on the skin is barely perceptible, being similar in nature to hardened candle wax; there is virtually no oily or greasy feel.

1. An emulsion composition for skin application comprising an aqueous component, one or more water-insoluble organic ingredients totaling at least about 15 percent by weight of the composition, and about 1 to about 10 percent by weight of porous silica microspheres having an average particle size between about 5 μm and about 20 μm.

2. The emulsion composition of claim 1, wherein the water-insoluble organic ingredients comprise at least about 20 percent by weight.

3. The emulsion composition of claim 1, wherein the water-insoluble organic ingredients comprise at least about 25 percent by weight.

4. The emulsion composition of claim 1, wherein the silica microspheres comprise about 2 to about 8 percent by weight.

5. The emulsion composition of claim 1, wherein the silica microspheres comprise about 3 to about 6 percent by weight.

6. The emulsion composition of claim 1, wherein the water-insoluble organic ingredients comprise sunscreen active ingredients, in an amount up to about 50 percent by weight of the composition.

7. The emulsion composition of claim 1, wherein the water-insoluble organic ingredients comprise sunscreen active ingredients, in an amount about 10 to about 35 percent by weight of the composition.

8. An emulsion composition for skin application comprising an aqueous component, a sunscreen component totaling at least about 10 percent by weight of the composition, and about 1 to about 10 percent by weight of porous silica microspheres having an average particle size between about 5 μm and about 20 μm, wherein a total amount of water-insoluble organic ingredients comprises at least about 25 percent by weight of the composition.

9. The emulsion composition of claim 8, wherein the sunscreen component comprises two or more sunscreen active ingredients.

10. The emulsion composition of claim 8, wherein the sunscreen component comprises up to about 35 percent by weight of the composition.

11. The emulsion composition of claim 8, wherein the silica microspheres comprise about 2 to about 8 percent by weight.

12. The emulsion composition of claim 8, wherein the silica microspheres comprise about 3 to about 6 percent by weight.

13. An emulsion composition for skin application, formed by combining: an aqueous component containing porous silica microspheres having an average particle size between about 5 μm and about 20 μm and comprising about 2 to about 8 percent by weight of the composition; and an oil component containing one or more organic sunscreen ingredients totaling at least about 10 percent by weight of the composition; wherein the oil component comprises at least about 25 percent by weight of the composition.

14. The emulsion composition of claim 13, wherein the oil component comprises two or more organic sunscreen active ingredients.

15. The emulsion composition of claim 13, wherein the silica microspheres comprise about 3 to about 6 percent by weight.

16. The emulsion composition of claim 13, wherein the organic sunscreen ingredients comprise about 10 to about 35 percent by weight.

17. An emulsion composition comprising: an aqueous component containing porous silica microspheres having an average particle size between about 5 μm and about 20 μm and comprising about 2 to about 8 percent by weight of the composition; and an oil component comprising two or more sunscreen ingredients selected from the group consisting of homosalate, oxybenzone, octisalate, avobenzone, and octocrylene the sunscreen ingredients being present in amounts that establish an SPF value at least about 45 for the composition.

18. The emulsion composition of claim 17, wherein the silica microspheres comprise about 3 to about 6 percent by weight.

19. An emulsion composition comprising: an aqueous component containing porous silica microspheres having an average particle size between about 5 μm and about 20 μm and comprising about 2 to about 8 percent by weight of the composition; and an oil component comprising two or more sunscreen ingredients selected from the group consisting of homosalate, oxybenzone, octisalate, avobenzone, and octocrylene, the sunscreen ingredients being present in amounts that establish an SPF value at least about 30 for the composition.

20. The emulsion composition of claim 19, wherein the silica microspheres comprise about 3 to about 6 percent by weight.

21. An emulsion composition comprising: an aqueous component containing porous silica microspheres having an average particle size between about 5 μm and about 20 μm and comprising about 2 to about 8 percent by weight of the composition; and an oil component comprising two or more sunscreen ingredients selected from the group consisting of homosalate, oxybenzone, octisalate, avobenzone, and octocrylene, the sunscreen ingredients being present in amounts that establish an SPF value at least about 15 for the composition.

22. The emulsion composition of claim 21, wherein the silica microspheres comprise about 3 to about 6 percent by weight.
